(19) United States
(12) Patent Application Publication
(10) Pub. No.: US 2017/0072012 A1
(43) Pub. Date: Mar. 16, 2017

(54) USE OF GSMTX4 TO REDUCE ISCHEMIA REPERFUSION INJURY

(71) Applicant: The research Foundation for The State University of New York, Amherst, NY (US)

(72) Inventors: Frederick SACHS, Buffalo, NY (US); Ji LI, Williamsville, NY (US); Tom SUCHYNA, Amherst, NY (US); Jinli WANG, Buffalo, NY (US); Philip GOTTLIEB, Buffalo, NY (US)

(21) Appl. No.: 15/122,026

(22) PCT Filed: Mar. 9, 2015
(86) PCT No.: PCT/US2015/019388

§ 371 Date: Aug. 26, 2016

Related U.S. Application Data
(60) Provisional application No. 61/949,722, filed on Mar. 7, 2014.

(51) Int. Cl. A61K 38/17; A61L 29/16; A61K 9/00; A61L 31/16

(52) U.S. Cl. CPC A61K 38/1767; A61L 31/16; A61L 29/16; A61K 9/0019; A61L 2300/252

(57) ABSTRACT

The present invention provides methods for the inhibition of reperfusion injury by treatment with Grammastola spatulata mechanotoxin4 (GsMTx4). The GsMTx4 may be delivered to the ischemic tissue in an individual by any means prior to, during or after ischemia reperfusion injury.

### CROSS REFERENCE TO RELATED APPLICATIONS

This application claims priority to U.S. provisional application No. 61/949,722, filed on March 7, 2014, the disclosure of which is incorporated herein by reference in its entirety.

### STATEMENT REGARDING FEDERALLY SPONSORED RESEARCH

This invention was made with government support under Grant No. AG028163 from the National Institutes of Health. The government has certain rights in the invention.

### BACKGROUND OF THE DISCLOSURE

In economic terms, in 2011, Acute Myocardial Infarction (AMI) was one of the top five most expensive medical conditions seen during inpatient hospitalizations in the U.S., with an aggregate cost of approximately $11.5 billion for 612,000 hospital stays. So far, all clinical use of drugs including beta blockers, calcium channel blockers and vasodilators can significantly reduce morbidity of AMI patients, but not significantly decrease mortality. Therefore, both scientists and clinicians are working to find better drugs or other therapies that could reduce both morbidity and mortality.

Reperfusion of ischemic cardiac tissue by percutaneous coronary intervention has become a routine treatment for acute ST-elevation myocardial infarction (STEMI). However, restoring blood flow to ischemic cardiac tissue can lead to injury of cardiac myocytes that were viable right before reperfusion (Hausenloy et a;. J Clin Invest, 2013. 123(1):p. 92-100, Turer et al., Am J Cardiol, 2010. 106(3):p. 360-8). This ischemia reperfusion injury (IRI) has gained significant interest in recent years because the procedure can cause tissue death that may account for up to 50% of the final myocardial infarct (MI) area.

Reperfusion injury follows the restoration of blood flow to anoxic tissue. For the period 2005-2008 in the United States, the median mortality at 30 days was 16.6% with a range from 10.9% to 24.9%. Using variables available in the emergency room, people with a higher risk of adverse outcome can be identified. One study found that 0.4% of patients with a low-risk profile died after 90 days, whereas in high-risk people it was 21.1%. For example in India, ischemic heart disease (AMI) had become the leading cause of death by 2004 accounting for 1.46 million deaths (14% of total deaths) and deaths due to ischemic heart disease were expected to double during 1985-2015.

The putative causes of IRI are varied, including cation imbalances elevation of reactive oxygen species (ROS), microvasculature obstruction due to endothelial and myocyte swelling and impaired NO vasodilation, inflammatory cell invasion and autophagy. Despite substantial progress in understanding the mechanisms of IRI, chemical interventions meant to treat side effects have been largely disappointing (Hausenloy et al., 2013, and Turer et al., 2010).

### SUMMARY OF THE DISCLOSURE

The present disclosure provides the use of the peptide, Grammostola spatulata Mechanotoxin4 (GsMTx4) and variants thereof, to inhibit reperfusion injury including reperfusion-induced arrhythmia. Due to inhomogeneous local mechanical stresses, mechanoselective channels may be significant contributors to the cation imbalance observed during ischemic reperfusion injury across multiple systems.

In one embodiment, this disclosure provides a method for reducing ischemic reperfusion injury. In one embodiment, this disclosure provides a method for reducing ischemic reperfusion injury to cardiac tissue. The method comprises administering to an individual in need of treatment, a therapeutically effective dose of GsMTx4. The peptide may be administered prior to an expected IRI, during IRI or after IRI. In one embodiment, the therapeutically effective dose of GsMTx4 is administered within 48 hours of an expected IRI or within 24 hours of the occurrence of

### BRIEF DESCRIPTION OF THE FIGURES

FIG. 1. GsMTx4-D was administered to mice by IV bolus and subcutaneous injection and its concentration in the plasma and heart were monitored by LC-MS/MS over 24 hrs. The peptide spiked in <5 min after IV injection in both plasma and heart. Subcutaneous injections provides lower absorption and peaked at about ˜2 hrs in both plasma and heart. The plasma concentration decayed rapidly for both mechanisms of injection, while the heart concentration reached a steady level that lasted the length of the study suggesting a strong association with the tissue and negligible degradation.

FIG. 2. Acute administration of GsMTx4-D reduces myocardial infarction during FR. Hearts were subjected to 20 min ischemia followed by 4 h of reperfusion. Different doses of GsMTx4-D (0.1, 1, 10 mg/kg), verapamil (0.4 mg/kg) or vehicle (saline 50 μL) were administered through tail vein injection 5 min before initiating reperfusion. At the conclusion of reperfusion, the tissue was dual stained with TTC and Evans Blue. The white area shows the myocardial infarction, red and white areas show area at risk (AAR), and the blue area shows the non-ischemic tissue. Panel A shows representative sections of myocardial infarction. Panel B (left chart) shows the ratio of area at risk (AAR) to total myocardial area and Panel B (right chart) shows the ratio of infarct area to AAR. Values are means±SE from 5 independent experiments. * denotes P<0.05 vs. vehicle, and † denotes P<0.05 vs. verapamil.

FIG. 3. Daily administration of GsMTx4-D reduces myocardial infarction during I/R and improved heart function 24 hrs after reperfusion. GsMTx4-D (50 mg/kg) or vehicle (saline 50 μL) were administered daily IP for 2 days prior to surgery. Ischemia was produced for 20 min, followed by 24 h of reperfusion. At the conclusion of reperfusion, mice were subjected to left ventricular catheterization to acquire real-time pressure-volume parameters. Following these measurements the tissue was dual stained with TTC and Evans Blue dyes. Panel A shows representative sections of hearts from vehicle and GsMTx4-D injected mice. The coloring shows the myocardial infarction area, the AAR and the non-ischemic tissue as described in FIG. 2. Graphical representations of the ratio of area at risk (AAR) to total myocardial area and the ratio of infarct area to AAR are shown in B and C respectively. Values are means±SE from 5 independent experiments. GsMTx4-D significantly reduced the infarcted fraction of the AAR (** denotes P<0.01). Cardiac function parameters are shown in (D—heart rate), (E—stroke volume), (F—cardiac output), (G—ejection fraction), (H—end systolic volume) and (I—end diastolic volume). Values are means±SE from 5 (sham and GsMTx4-D) to 6 (vehicle) independent experiments. Statistically significant changes in vehicle injected animals vs sham experiments is denoted as * (P<0.05) and ** (P<0.01), and statistically significant improvements for GsMTx4-D over vehicle injected mice is denoted † (P<0.05) and †† (P<0.01).

FIG. 4. GsMTx4-D suppressed FR induced arrhythmias. (A) Representative ECG: baseline, ischemia and reperfusion. Hearts were subjected to 20 min ischemia followed by 4 hr of reperfusion. ST-segments were elevated during coronary occlusion and the T-wave inverted during reperfusion. (B) Arrhythmias occurring within 15 min of initiating reperfusion were counted to measure suppression, and examples of these events are indicated by arrows above the data records. (C) Summary of arrhythmia incidence including supraventricular and ventricular premature beat. (D) Incidence of the more severe ventricular premature beat. (E) Mean heart rate before ischemia. (F) Mean heart rate during 15 min of reperfusion. Values are means±SE from 5 to 7 independent experiments (vehicle n=7, verapamil n=6, GsMTx4-D 0.1 mg/kg n=5, 1 mg/kg n=6 and 10 mg/kg n=5). Statistical significance is denoted as * (P<0.1) and ** (P<0.05) vs. vehicle.

### DESCRIPTION OF THE DISCLOSURE

The present disclosure describes the use of GsMTx4 to reduce ischemic reperfusion injury. Without being bound by any theory, our work indicates that cation selective mechanosensitive ion channels (MSCs) may be pertinent during reperfusion.

GsMTx4 is a peptide that may have 34 or 35 amino acids as described herein. This peptide selectively inhibits gating of cationic MSCs and is active as either the L or D enantiomeric forms (Suchyna et al., Nature, 2004. 430 (6996), p. 235-240; Suchyna et al., Journal of General Physiology, 2000. 115: p. 583-598). The term GsMTx4 is used herein to refer to GsMTx4-L, GsMTx4-D or a combination of GsMTx4-L and GsMTx4-D. Accordingly, both L and D enantiomers of GsMTx4 and its variants can be used for the present invention.

GsMTx4 is made synthetically under GMP conditions and is effective when made from L amino acids (natural form) or D amino acids. GsMTx4 can be made by the procedures set forth in U.S. Pat. Nos. 7,125,847 and 7,259,145, which are hereby incorporated by reference. Briefly, L-GsMTx4 can be prepared by purification of the Grammostola spatulata venom. The venom is commercially available (Spider Pharm, PO Box 1090 Yarnell, Ariz. USA (928-427-6589)). or may be elicited from the spider by standard techniques such as electrical stimulation. GsMTx4 can be isolated from spider venom by serial fractionation using standard chromatographic techniques, such as reverse phase high performance liquid chromatography. GsMTx4 can also be prepared by chemical synthesis using automated or manual solid phase methods (e.g., Fmoc chemistry or automated synthesis). Depending upon quantitative yields, production of the linear reduced peptide can be performed in either a single process or in two different processes followed by a condensation reaction to join the fragments. A variety of protecting groups can be incorporated into the synthesis of linear peptide so as to facilitate isolation, purification and/or yield of the desired peptide. Protection of cysteine residues in the peptide can be accomplished using protective agents such as triphenylmethyl, acetamidomethyl and/or 4-methoxybenzyl group in any combination.

The peptide GsMTx4 and its variants may also be prepared by recombinant DNA technology. A DNA sequence coding for the peptide is prepared, inserted into an expression vector and expressed in an appropriate host cell. The expressed peptide can then be purified from the host cells and/or culture medium. Methods for preparing DNA coding for the peptide and expression of DNA are well known to those skilled in the art and are found for example, in Sambrook et al., (1989) Molecular Cloning: A Laboratory Manual, Cold Spring Harbor, N. Y., S. L. Berger and A. R. Kimmel, Eds., Guide to Molecular Cloning Techniques: Methods in Enzymology, vol 152, Academic Press, San Diego, Calif., 1987, and in E. J. Murray, Ed., Gene Transfer and Expression Protocols: Methods in Molecular Biology, vol 7, Humana Press, Clifton, N. J., 1991. In addition, the cloning of a cDNA encoding the GsMTx4 peptide is also disclosed.

GsMTx4 can be prepared as a pharmaceutical composition by incorporation with a pharmaceutically acceptable carrier or diluent. GsMTx4 can be formulated into tablets, capsules, caplets and the like. Suitable carriers for tablets include calcium carbonate, starch, lactose, talc, magnesium stearate and gum acacia. GsMTx4 can also be formulated for oral or parenteral (e.g., intravenous, subcutaneous or intramuscular) administration in aqueous solutions, aqueous alcohol, glycol or oil solutions or emulsions. GsMTx4 can also be formulated for inhalation by encapsulation to facilitate alveolar absorption as has been done for insulin (Inhale Therapeutic Systems, San Carlos, Calif., www.Inhale.com). Pharmaceutical compositions suitable for such routes of administration are well known in the art. For example, suitable forms and compositions of pharmaceutical preparations can be found in Remington's Pharmaceutical Science. Thus, GsMTx4 can be administered orally, parenterally, intravenously, intramuscularly, intrathecally or intranasally. The peptide may also be locally applied via the tip of a catheter or other devices coming into contact with the heart during invasive procedures.

In the present disclosure, we show that GsMTx4 to be efficacious in protection from reperfusion injury and reperfusion-induced arrhythmia. Reperfusion injury and reperfusion-induced arrhythmia can occur in AMI and in many other pathological states, such as acute traumatic injury (e.g. crushing), angina, and cardiac, transplantation and other surgeries that require reperfusion. Our data supports the theory (without being bound thereby) that reduced cation flux through mechanosensitive channels during reperfusion events is advantageous to tissue viability.

When the ischemic event or injury such as crushing has occurred prior to presentation for surgery, it is preferable to administer the GsMTx4 as soon as possible, such as in an ambulance or emergency room prior to surgery. GsMTx4 can also be administered prior to releasing a crush victim from the crushing object.

In one embodiment, the subject treated with GsMTx4 is a mammal, such as a human. In one embodiment, the subject treated with GsMTx4 is a non-human animal including domestic animals.

In one embodiment, GsMTx4 is administered at a minimum concentration of about 1 mg/kg prior to reperfusion. In one embodiment, GsMTx4 is administered at a dosage between 0.1-20 mg/kg via an intravenous route. If administered via the subcutaneous or intraperitoneal routes, the dosage can be increased. For example, the dose can be increased to 10 times the dose of the IV route.

In one embodiment, GsMTx4 is administered to achieve a circulating level of from 0.5 to 5.0 μM and all values therebetween to the tenth decimal place. In one embodiment, GsMTx4 is administered to achieve circulating levels from 0.1 to 20 μM.

In addition to GsMTx4 being administered i) prior to ischemia and reperfusion and ii) during ischemia but prior to reperfusion, GsMTx4 can also be administered a short time after reperfusion following ischemia. Thus situation can arise after a crush victim has been released from the crushing object. Without being bound by any theory, GsMTx4 is effective post-ischemia and post-reperfusion since it can reduce inflammation caused by ischemia and reperfusion, reduce reactive oxygen species and balance calcium homeostasis.

The window of opportunity in which the administration of GsMTx4 will be effective post-ischemia and post-reperfusion can be determined by those of skill in the art. In one embodiment, GsMTx4 is administered anywhere from a time that is 10 days prior to IRI to a time that is 2 days after IRI, including during IRI. In one embodiment, it is administered anytime from 48 hours prior to IRI to a time that is 24 hours after IRI. In one embodiment, it is administered just prior to (such as a 1-2 minutes), or during IRI. For example, the peptide can be delivered simultaneously with release of occlusion of a blocked blood vessel. For example, an interventional device (stent, catheter, balloon and the like) may be used to deliver and release GsMTx4.

The present peptide, GsMTx4 and/or its variants can be used in combination with other therapeutic approaches to treat reperfusion injury and reperfusion-induced arrhythmia.

The peptide GsMTx4, as used herein, has the sequence GCLEFWWKCNPNDDKCCRPKLKCSKLFKLCNFSF (SEQ ID NO:1). This peptide is referred to in this application as GsMTx4 or GsMTx4(34 mer). Substitutions of the last three C-terminal amino acids can be made. Any such amino acid substitution that does not adversely affect the capability of the peptide(s) to inhibit mechanoselective ion channels and cation-permeable conductance in a cell can be made and peptides comprising such substitutions are encompassed within the present invention. Such substitutions can be readily identified by those skilled in the art. In this regard, several substitutions were made and these were found not to affect the properties of the peptide. Therefore, in one embodiment, the peptide of the present invention is GCLEFWWKCNPNDDKCCRPKLKCSKLFKLCNXaaXaaXaa (SEQ ID NO:2). In one embodiment, the amino acid at the 32^{nd} position is Y (instead of F) thus providing the sequence GCLEFWWKCNPNDDKCCRPKLKCSKLFKLCNYSF (SEQ ID NO:3). In another embodiment, the serine at the 33^{rd} position can be changed to cysteine, providing the sequence GCLEFWWKCNPNDDKCCRPKLKCSKLFKLCNFCF (SEQ ID NO:4), and if desired, the cysteine can be derivatized with reporters such as fluorescein. In another embodiment, the phenylalanine at the 34^{th} position can be changed to serine and/or another amino acid can be added as the 35^{th} amino acid providing the sequence GCLEFWWKCNPNDDKCCRPKLKCSKLFKLCNFSSA (SEQ ID NO:5).

Each peptide described herein for use in the compositions and methods of the invention can comprise or consist of the amino acid sequence provided for it. The peptides presented herein may have an N-terminal amide. In one embodiment, the present invention provides pharmaceutical compositions comprising a peptide which is 34, 35 or 36 amino acids long and comprises the sequence of SEQ ID No. 2. In another embodiment, the pharmaceutical composition comprises a peptide which is 34, 35 or 36 amino acids long and comprises the sequence of SEQ ID NO:1. In another embodiment, the pharmaceutical composition comprises a peptide which is 34, 35, 36 amino acids long and comprises the sequence of SEQ ID NO:3, 4 or 5.

The amount of GsMTx4 or its variants in the pharmaceutical composition can be determined by empirical methods. Those skilled in the art will recognize that the dosage administered to a particular individual will depend on a number of factors such as the route of administration, the duration of treatment, the size and physical condition of the individual, and the patient's response to the peptide. The lack of any measurable toxicity of GsMTx4 in animal studies provides flexibility for designing a broad range of dosage regimens. The term “treatment” as used herein means reducing the severity of one or more symptoms of or associated with reperfusion injury.

In one embodiment, the method of this disclosure may be used for reducing ischemia reperfusion injury that is caused by reduction or cessation of blood flow to any vascular bed in the body.

In one embodiment, GsMTx4 may be delivered to site of IRI via interventional devices. In one embodiment, an interventional device may be a catheter, including injection catheters and balloon catheters. In one embodiment, the interventional device may be a stent, including permanent and biodegradable stents, peripheral stents and coronary stents, vascular grafts and conduits vascular shunts, biological conduits, valve grafts, and the like. In one embodiment, interventional devices may be polymeric delivery devices. These devices could deliver GsMTx4 as they are being used for reversal of occlusion that is the cause of ischemia, or they may be used to deliver the peptide without the occlusion reversal function. By this method, GsMTx4 may be delivered to ischemic areas, including ischemic myocardial and brain tissue at and/or downstream of the implantation site when positioned directly at or near a site of a previously occluded blood vessel. The delivery of an anti-ischemic agent locally at the ischemic injury site will improve the viability of the cells by reducing ischemic injury to the cells (such as myocardial cells or brain cells) including reperfusion injury which may occur upon return of blood flow to the ischemic tissue. For example, when reperfusion is performed by angioplasty, a stent is often delivered to the reopened occlusion site. GsMTx4 may be delivered to the site as a releasable composition on the stent.

For delivery of GsMTx4 on site by an interventional device, the device (such as a stent, the tip of a catheter, or a balloon) may be coated with a releasable composition comprising GsMTx4. Such compositions are known in the art. For example, release of agents in a polymeric coating on an implantable medical device are described in U.S. Pat. Nos. 6,273,913 and 6,712,845. Generally, the stent may be coated with GsMTx4 in a polymer matrix, Coating may be carried out by methods such as, for example, dipping, spraying, precipitation, and the like. In one embodiment, GxMTx4 may also be directly adsorbed on to the interventional device without a polymer. In one embodiment, an interventional may be provided with holes or recesses which can provide means for sustained release of the peptide.

In one aspect, this disclosure provides an interventional medical device, such as a stent or a catheter to which is releasably attached, GsMTx4. In one embodiment, the GsMTx4 that is releasably attached to the catheter or stent is GsMTx4-D.

In on embodiment, this disclosure provides a method of delivery of GsMTx4 to, or near, the site of an ischemic injury comprising inserting an interventional device into a patient (such as via any suitable blood vessel including femoral artery) in need of treatment and then allowing the release of the peptide at or near the site of the ischemic injury.

The invention is further described through the following examples, which are meant to illustrative and not limiting.

### EXAMPLE 1

We injected GsMTx4-D into mice to determine its effect on ischemic reperfusion injury in mouse heart. Both pretreatment of the mice by a slower absorption method of intraperitoneal (IP) administration, and the more rapid absorption method of intravenous (IV) injection during ischemia were tested. Ischemia was induced in mice by coronary artery ligation and then reperfused by release of the ligature. GsMTx4-D substantially reduced cardiac infarct area by both pretreatment for two days and acute IV administration during an ischemic event. These cardioprotective properties extended up to 24 hrs in the pretreated animals, which includes stages when “late phase” apoptotic cell death begin occurring.

There were no overt signs of toxicity in the heart during these studies. In fact, after acute IV injection treatments there was a reduction in cardiac arrhythmias, and increased cardiac output after the longer term pretreatments. GsMTx4-D showed substantially enhanced improvement over the voltage gated channel blocker Verapamil suggesting different target channels.

### Materials and Methods

### Experimental Animals and Cardiac Phamacokinetic (PK) Analysis

For single-dose pharmacokinetic studies, 7-9 week old female CD-1 mice were obtained from Charles River Laboratories, Inc. by Covance Laboratories Inc., Greenfield, Ind., USA. For subcutaneous injection, GsMTx4-D was dissolved at a concentration of 7.5 mg/ml in phosphate buffered saline pH 7.4 and injected subcutaneously into the back of mice at a net dose of 50 mg/kg. For intravenous (IV) administration into the tail vein, 1.5 mg/ml GsMTx4-D was used to achieve a dose of 5 mg/kg. Animals were sacrificed at 5, 15, 30, 60, 120, 480, 960 and 1920 min after injection, and blood and heart tissue samples were harvested. Animals were checked for death or morbidity during the whole trial. At the conclusion of each trial period, mice were euthanized by CO2 inhalation for 10 min. Blood samples were treated with sodium heparin and centrifuged to obtain plasma. There were cohorts of 3 mice per time point, and tissue samples from the 3 animals were combined from each time point to create average samples. Samples were sent to Custom Biologics, Mississauga, ON, Canada to measure the amount of GsMTx4 by liquid chromatography tandem mass spectrometry (LC/MS/MS) in the tissue samples. A reference standard was included in each sample consisting of a functional mutant of GsMTx4-D (F34A). All procedures in this protocol are in compliance with the U.S. Department of Agriculture's (USDA) Animal Welfare Act (9 CFR Parts 1, 2, and 3); the Guide for the Care and Use of Laboratory Animals: Eighth Edition, (Institute for Laboratory Animal Research, The National Academies Press, Washington, D.C., 2010); and the National Institutes of Health, Office of Laboratory Animal Welfare. Whenever possible, procedures in this study were designed to avoid or minimize discomfort, distress, and pain to animals. For ischemic reperfusion (I/R) testing, we used male C57B1/6J mice (4-6 months of age) obtained from Jackson Laboratories (Bar Harbor, Minn.). All animal protocols in this study were approved by the Institutional Animal Care and Use Committee of the University at Buffalo-State University of New York.

### I/R Model In Vivo

Acute GsMTx4-D treatment: All animal protocols in this study were approved by the Institutional Animal Care and Use Committee of the University at Buffalo-State University of New York. C57B1/6J mice were anesthetized with 80 mg/kg of sodium pentobarbital (Sigma, St. Louis, Mo.) via intraperitoneal injection (IP), intubated and ventilated with a respirator (Harvard apparatus, Holliston, Mass., USA) 17. Upon absence of a pedal reflex, surgery was performed under a stereomicroscope. Core body temperature was maintained at 37° C. with a heating pad. After a left lateral thoracotomy, the left anterior descending coronary artery (LAD) was occluded just underneath the left atrium for 20 min with an 8-0 nylon suture. Shorter ischemia times more readily produce nonreversible infarct areas in mice than in larger animals, and we found that 20 min of ischemia was sufficient to produce a highly reproducible infarct area. A small piece of gauze was used to prevent arterial injury. After 20 min the ligature was removed and the tissue was reperfused for up to 4 h before euthanizing. Three different doses of GsMTx4-D (0.1, 1, 10 mg/kg), verapamil (0.4 mg/kg) or vehicle (saline 50 μL) were administered via tail vein injection 5 min before reperfusion (during induced ischemia). ECGs confirmed the hallmark ischemic ST segment elevation during coronary occlusion (AD Instruments, Colorado Springs, CO, USA). Each group included 5 independent experiments. At the conclusion of the study, mice were euthanized by IP injection of 270 mg/kg pentobarbital. GsMTx4-D pretreatment: To evaluate the effect of GsMTx4-D pretreatment, GsMTx4-D (50 mg/kg) or vehicle (saline 50 μL) were administered via IP injection once daily for 2 days prior to surgery. Ischemia was then elicited with the same 20 min coronary occlusion followed by 24 h reperfusion. Following reperfusion, the chest was closed in layers. A drop of lidocaine was put on the suture site and the mouse was slowly weaned off the ventilator when they resumed spontaneous breathing. The mouse was returned to its cage and kept warm and medicated for pain (buprenorphine). Each group included 5 independent experiments.

### Heart Rhythm Analysis

During reperfusion we continuously recorded ECGs, from which mean heart rate, PR, QRS, and QT intervals were derived for each animal. We evaluated the incidence of arrhythmias by counting the incidences of arrhythmias including ventricular and supraventricular premature beats by summing the number of episodes based on limb lead recordings for 15 min after reperfusion. Ventricular premature beats are considered more severe. Each group contained 5 to 7 independent experiments (vehicle n=7, verapamil n=6, GsMTx4-D 0.1mg/kg n=5, 1 mg/kg n=6 and 10 mg/kg n=5).

In vivo Pressure-Volume Relationships

In order to determine hemodynamic differences, we measured the pressure-volume (P-V) relationships in real time in 3 groups of mice, including a sham group that was exposed to the open heart operation but not FR, and the two I/R (20min ischemia/24h reperfusion) induced groups pretreated with either vehicle or GsMTx4-D. To measure the P-V relationships, mice were anesthetized with 4% isoflurane and 100% O₂, quickly intubated and ventilated with a respirator. The amount of isoflurane was slowly reduced to 1-1.5% until the pedal reflex was no longer observed. A horizontal incision was made inferior to the sternum and oxphoid process. The rib cage was slightly retracted and the diaphragm was gently cut away to attain an apical view of the myocardium. A 27-gauge needle was inserted into the apex of the LV free wall to create a guide-hole. A 1.2-French microtransducer pressure-volume impedance catheter (Scisense, London, ON, CA) was then inserted into the lumen of LV to assess real-time LV function. Hemodynamic parameters such as heart rate, cardiac output, and ejection fraction were measured and calculated with the iWorx software (Dover, N.H.). Each group included 5 (sham and GsMTx4-D) to 6 (vehicle) independent experiments.

### Infarct Size Measurement

To measure infarct size, hearts were harvested and excised for dual staining at the conclusion of reperfusion. The non-necrotic tissue in the ischemic region was stained red with 2,3,5- triphenyltetrazolium (TTC) and non-ischemic regions were stained blue with Evans blue. The hearts were fixed and sectioned into 1 mm slices, photographed with a Lexica MZ95 microscope and analyzed with NIH ImageJ software (http://imagej.nih.gov/ij/) 19. The infarct size was calculated as the ratio of the percentage of myocardial necrosis to the ischemic Area At Risk (AAR).

Statistical analysis

Data are expressed as means±SE. Statistical significance was analyzed with either a student's t-Test or one-way ANOVA where appropriate. For multi-pairwise comparisons the Tukey post hoc test was performed to measure individual group differences of interest. The F and a significance levels of 0.1, 0.05 and 0.01 are indicated.

### Results

### GsMTx4-D Heart and Plasma Pharmacokinetics

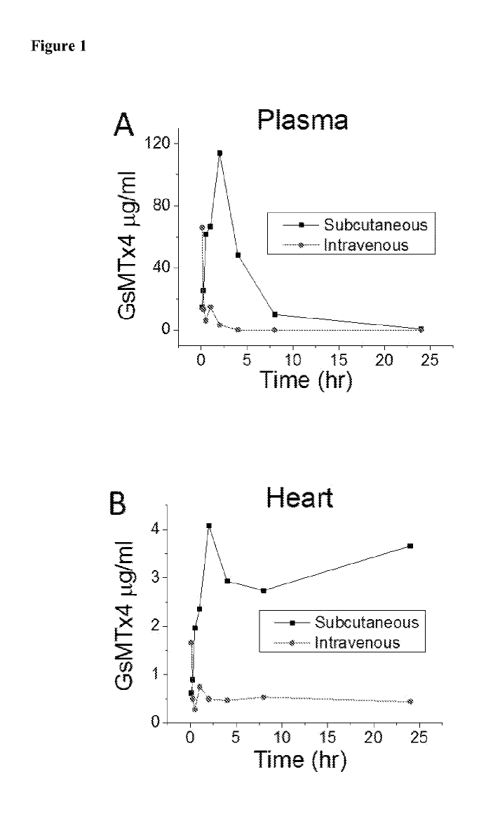

To provide a frame of reference for concentration and timing, we compared IV (fast absorption) to subcutaneous (slow absorption) administration and determined the distribution of the peptide in the heart and plasma over 24 hrs using LC/MS/MS (FIG. 1). Mice were sacrificed at different times after injection and the plasma (FIG. 1A) and heart (FIG. 1B) concentrations sampled. The concentration in both tissues had a transient peak followed by a low steady state. The samples from the 3 mouse cohorts at each time point were mixed and averaged. No mice died during the study, nor did any show abnormal behavior (i.e. animals were active and did not show signs of morbidity). IV injection of 5 mg/kg GsMTx4-D produced an early spike in plasma concentration (>60 mg/ml at 5 min) that decayed within 30 min. The heart showed a significantly smaller spike (<2 mg/ml at 5 min) that decayed to ˜0.5 mg/ml. We used 1-5 μM (4.1 mg/ml is 1 μM) as our target concentration for treatment. IV injection of 5 mg/kg GsMTx4-D produced plasma concentrations within 5 minutes that were substantially above the target concentration while whole heart concentrations were somewhat lower than this range. Other ranges may be used for treatment, such as about 1.5-4.5 μM, about 2-4 μM, about 2.5-3.5 μM, about 1-4 μM, about 1-3 μM and about 1-2 μM. The whole heart concentration was lower than the target range within 5 minutes of IV injection, but the net plasma concentrations were acceptable.

Based on the slower distribution by subcutaneous administration, we injected 10 times the concentration of GsMTx4-D as that used in IV injection. The concentrations increased more slowly in both plasma and heart, peaking in ˜2 hrs, although we could reliably detect it in both tissues within 5 minutes. The plasma concentration declined after 2 hours and was nearly undetectable by 24 hrs, while the heart concentration remained high suggesting higher affinity. Twenty four hours after a single 50 mg/kg subcutaneous injection, the heart concentration was near the target range. Without being bound by any theory, we believe that this is likely due to deeper infiltration into the tissue, and possibly higher affinity associations. Twenty four hours after one 50 mg/kg subcutaneous injection the heart concentration was near the target range.

### GsMTx4-D Reduces Myocardial Infarction During I/R

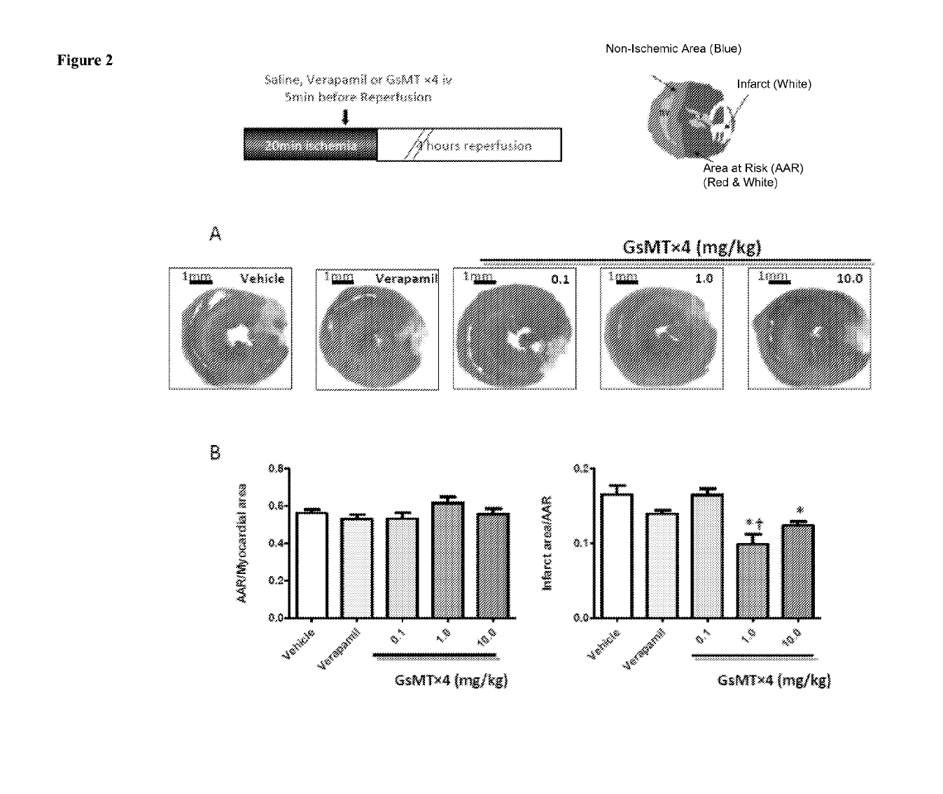

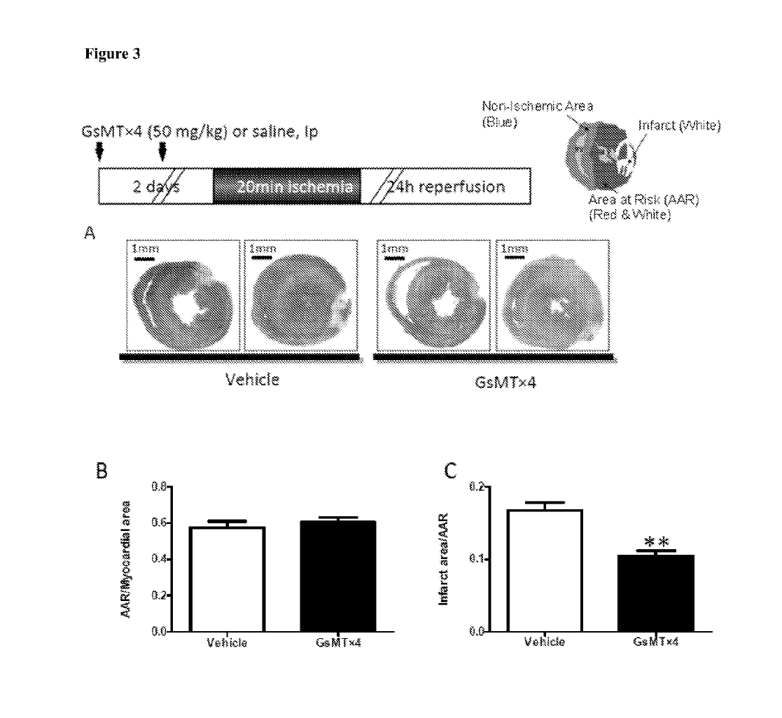

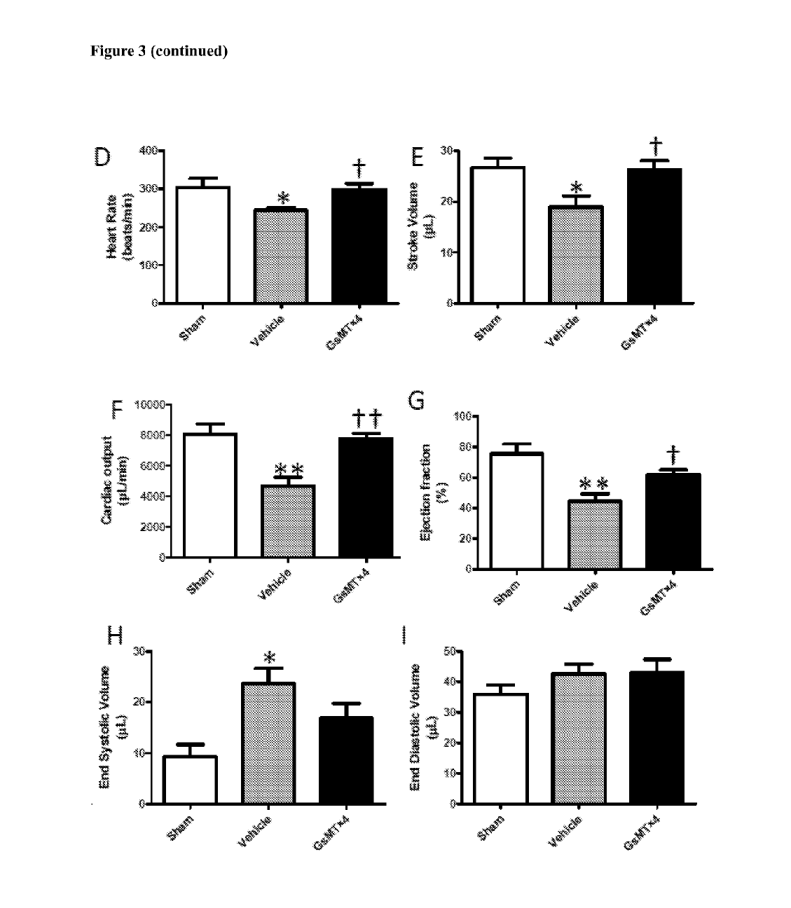

To test whether GsMTx4-D protected the heart against reperfusion injury, we first tested three different doses of the peptide. The positive control was verapamil, a voltage-gated calcium channel inhibitor with established cardioprotective effects on IRI. C57B1/6 mice were subjected to 20 min occlusion of the LAD followed by 4 hours of reperfusion. Three different concentrations of GsMTx4-D (0.1, 1 and 10 mg/kg), verapamil (0.4 mg/kg), or vehicle (saline) were administered through tail vein injection 5 min before reperfusion. There was no death or morbidity observed. At the conclusion of reperfusion, the hearts were stained with TTC and Evans Blue (FIG. 2A). The ratios of the AAR to total myocardial area were equal among the five cohorts (FIG. 2B), demonstrating the high degree of reproducibility for this procedure. GsMTx4-D at 1.0 mg/kg or higher dose significantly decreased myocardial infarction (9.9±1.3% at 1.0 mg/kg, 12.4±0.5% at 10 mg/kg; vs. 16.5±1.2% with vehicle, P<0.05 vs. vehicle). The verapamil group also had lower infarction size (14.0±0.4%), but the difference wasn't statistically significant. Thus, acute treatment with GsMTx4 at 1.0 or 10 mg/kg prior to reperfusion, showed better protection against infarct formation than verapamil. To evaluate whether pretreatment with GsMTx4-D could also protect the heart from I/R injury, GsMTx4-D (50 mg/kg) or vehicle (saline 50 μL) were administered daily via IP injection, for 2 days prior to I/R surgery. As before, ischemia was induced from 20 min LAD occlusion, but reperfusion was allowed to continue for 24 hrs. The chest was closed in layers, and animals were weaned off the ventilator when they resumed spontaneous breathing. Again, we did not detect any side-effects from GsMTx4-D treatments. The ratios of AAR to total myocardial area between the two groups were equal as determined by TTC and Evans Blue staining (FIG. 3A and B). GsMTx4-D decreased infarction size significantly (10.5±0.7% for GsMTx4-D 50 mg/kg IP vs. 16.8±1.1% for vehicle, P<0.01, FIG. 3C). This showed that the concentration of GsMTx4-D sustained in cardiac tissue 24 hrs after IP injection still significantly reduced infarction size, but had no adverse effects on normal behavior prior to ischemia induction. Additionally, the infarct area at 24 hrs is nearly identical to that observed at 4 hrs in the acute treatment experiments showing the infarct size is sustained. Our data indicated GsMTx4-D reduced myocardial I/R infarction size both in daily use and in emergent administration in FR models.

GsMTx4-D Improves Heart Function During I/R In vivo

To examine the effects of GsMTx4-D on hemodynamics, we compared the P-V relationships between the GsMTx4-D (50 mg/kg) and vehicle (saline 50 μL) in pretreated mice. After 20 min of ischemia followed by 24 h reperfusion, the left ventricle was catheterized to acquire real time P-V relationships (FIG. 3). Compared with sham-operated control animals, I/R significantly impaired pump function in vehicle control mice, including heart rate (243.9±6.9 bpm for vehicle vs. 304.0±23.6 bpm for sham, P<0.05), stroke volume (18.9±2.3 μl for vehicle vs. 26.7±1.9 μl for sham, P<0.05), cardiac output (4636±602 μl/min for vehicle vs. 8039±690 μl/min for sham, P<0.01) and ejection fraction (44.4 ±4.9% for vehicle vs. 75.5±6.4% for sham, P<0.01) (FIG.3D-I). In contrast, mice pretreated with GsMTx4-D showed improved heart rate (298.7±14.3 bpm, P<0.01), stroke volume (26.2±1.9 μl, P<0.1) and cardiac output (77580±321 μl/min, P<0.01) compared to vehicle. In addition, GsMTx4-D produced improvements in both end systolic and diastolic volumes (FIG. 3 H and I) that led to a significant improvement in ejection fraction (61.7±3.4%, P<0.05) (FIG.3 G). Clearly, GsMTx4-D improves heart function after FR.

### GsMTx4-D Reduce I/R Induced Arrhythmia

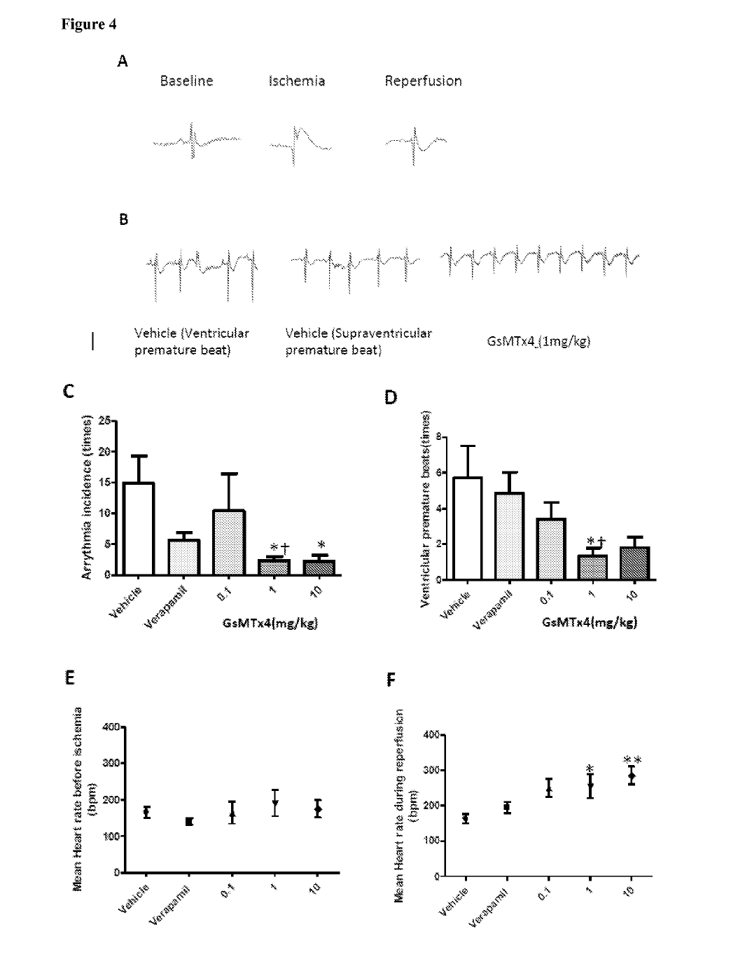

Heart rate and rhythm were monitored by ECG to confirm the hallmark ischemic ST-segment elevation during coronary occlusion, and the gradually increasing inversion of the T-wave during reperfusion (FIG. 4A). Since generation of arrhythmias is a good indicator of the level of I/R damage, we counted ventricular and supraventricular premature beats occurring within 15 min of reperfusion (FIG. 4B). No ventricular tachycardia was observed during 20 min ischemia and 15 min reperfusion in the mouse heart. The results showed that, while both verapamil and GsMTx4-D reduced total incidence of arrhythmias, IV injection of 1 and 10 mg/kg GsMTx4-D (2.3±0.7 and 2.2±1.0 incidence/15 min reperfusion respectively, P<0.1) 5 min before reperfusion, caused significant reduction at α=0.1 compared to vehicle (14.9±4.5 incidence/15 min reperfusion). Verapamil and 0.1 mg/kg GsMTx4-D (5.7±1.2 and 10.4±6 respectively) were not significantly different. Considering that ventricular premature beats are indicative of more severe damage, the incidence of this specific arrhythmia was analyzed separately (FIG. 4 D). We observed results similar to those for the total incidence of arrhythmias, but only GsMTx4-D at 1 mg/kg significantly decreased ventricular premature beats (1.3±0.4, 1 mg/kg GsMTx4-D vs.5.7±1.8, vehicle, P<0.1). There was no difference in mean heart rate before ischemia (FIG. 4E). However, during the 15 min of reperfusion, GsMTx4-D at 1 mg/kg and 10 mg/kg IV injection increased the mean heart rate significantly compared with vehicle (255.7±33.4, 1 mg/kg GsMTx4-D vs.162.9±13.2 vehicle, P<0.05; 286.1±25.0 10 mg/kg GsMTx4-D vs. vehicle, P<0.01) (FIG. 4F). There was no difference in PR, QRS or QT intervals after treatment in all groups (data not shown).

GsMTx4-D substantially reduced infarct size during FR whether by pretreatment (IP injection) or acute (IV) administration during the ischemic event. These cardioprotective properties lasted for at least 24 hrs in the pretreated animals, which includes stages when “late phase” apoptotic cell death begins to occur. The D form of GsMTx4 is non-antigenic, resistant to proteolytic degradation 24 and has a half-life >24 hrs. The half-life is long enough to potentially serve as preventative therapy for patients thought to be prone to an infarct. We observed no overt signs of toxicity in the heart during these studies, consistent with the lack of toxicity observed in earlier studies using the L form of GsMTx4 on cardiac tissue. GsMTx4-D reduced the incidence of cardiac arrhythmias and increased cardiac output and ejection fraction compared to vehicle control mice exposed to ischemia.

Based on the PK analysis, we predict the GsMTx4-D concentration in the heart at the time of reperfusion was 1-2 μM after two IP pretreatments, and <0.5 μM after the 1 mg/kg IV injection during ischemia. These concentrations are likely too low to significantly affect voltage gated channel properties.

GsMTx4-D improved all measures of IRI as shown by decreased infarct area, increased cardiac output and decreased incidence of arrhythmias. GsMTx4-D activity appears to be most pertinent when cells experience pathological stress since there is little effect on cardiac electrical or functional properties in the normal state. GsMTx4-D can be used in reperfusion events occurring with transplantation, angina, cardiac surgery 4 and traumatic injury.

While the invention has been described through illustrative examples, routine modifications of the various embodiments will be apparent to those skilled in the art and such modifications are intended to be within the scope of this disclosure.

1. A method of reducing ischemic reperfusion injury (IRI) comprising administering to an individual in need of treatment a composition comprising an effective amount of GsMTx4 in a pharmaceutically acceptable carrier at a time from 10 days prior to the TM to 2 days after the IM.

2. The method of claim 1, wherein the GsMTx4 is GsMTx4-D.

3. The method of claim 1 or 2, wherein the individual is identified as being at risk of having a reperfusion injury, and the GsMTx4 is administered prior to the IM.

4. The method of claim 1, wherein the peptide is administered intravenously or subcutaneously.

5. The method of claim 1, wherein the GsMTx4 is delivered via a catheter or a stent.

6. The method of claim 1, wherein the concentration of GsMTx4 in the circulating blood is from 0.1 to 20 μM.

7. The method of claim 6, wherein the concentration of GsMTx4 in the circulating blood is from 0.5 to 2 μM.

8. The method of claim 1, wherein the GsMTx4 is administered one or more times from 48 hours prior to IRI to 24 hours after IRI.

9. The method of claim 1, wherein the GsMTx4 is delivered during IRI.

10. A device for reducing ischemia reperfusion injury comprising a delivery device configured to be administered via a vascular route, wherein the delivery device has GsMTx4 releasably attached thereon.

11. The device of claim 10, wherein the device is a stent or a catheter.

12. The device of claim 10, wherein the GsMTx4 is GsMTx4-D.

13. The device of claim 10, wherein the delivery device is also used to open up an occluded blood vessel.


SEQUENCE LISTING (5 sequences)

SEQ ID NO: 1 - protein, 34 aa, Grammostola spatulata
GCLEFWWKCNPNDDKCCRPKLKCSKLFKLCNFSF

SEQ ID NO: 2 - protein, 34 aa, Grammastola spatulata
  misc_feature (32)..(34): Xaa can be any naturally occurring amino acid
GCLEFWWKCNPNDDKCCRPKLKCSKLFKLCNXXX

SEQ ID NO: 3 - protein, 34 aa, Grammastola spatulata
GCLEFWWKCNPNDDKCCRPKLKCSKLFKLCNYSF

SEQ ID NO: 4 - protein, 34 aa, Grammastola spatulata
GCLEFWWKCNPNDDKCCRPKLKCSKLFKLCNFCF

SEQ ID NO: 5 - protein, 35 aa, Grammastola spatulata
GCLEFWWKCNPNDDKCCRPKLKCSKLFKLCNFSSA